(19) United States
(12) Patent Application Publication
(10) Pub. No.: US 2015/0122251 A1
(43) Pub. Date: May 7, 2015

(54) KINK RESISTANT INTUBATION DEVICE

(71) Applicants: Arman Azhir, Fountain Valley, CA (US); Farzad Shaygan, Irvine, CA (US)

(72) Inventors: Arman Azhir, Fountain Valley, CA (US); Farzad Shaygan, Irvine, CA (US)

(21) Appl. No.: 14/072,058

(22) Filed: Nov. 5, 2013

(51) Int. Cl. A61M 16/04

(52) U.S. Cl. CPC A61M 16/0486
USPC 128/200.26

(57) ABSTRACT

A kink resistant endotracheal tube intubation device and a method for using the device are provided. The kink resistant endotracheal tube intubation device has a first tube portion in operable connection with a second tube portion. The first and second tube portions of the endotracheal tube intubation may be integrated into one piece or formed of two or more portions that are in operable attachment. The first tube portion is preferably substantially rigid and it has a lumen for dispensing gas or fluids, for example to anesthetize a patient.

### FIELD

The present disclosure relates to an intubation device and related medical devices that are used to control the flow of air, drugs, fluids, foods, or other substances into and/or out of the patient.

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art.

In the course of treating a patient, an intubation device or related medical device may be used to control the flow of air, drugs, fluids, foods, or other substances into and/or out of the patient. Accordingly, intubation tubes such as tracheal tubes are used to control the amount and flow of one or more of the these substances into or out of a patient.

Endotracheal tubes are also used to provide access to the upper airways for controlled ventilation, assisted ventilation, and as a conduit for anesthetic gases during surgical operations, in addition to postoperative airway management. Tracheal tubes may be designed so as to be either inserted through the oral or nasal passages of a patient. The choice of using an oral or nasal tracheal tube depends on the type of medical or surgical procedure to be performed. Oral tracheal tubes are generally preferred over nasal tracheal tubes because they are easier to place correctly in the trachea.

Prior to the instant invention, existing endotracheal tubes were available in different sizes with different diameters and lengths from which doctors may select the closest approximate size for a particular patient. An endotracheal tube with too large of an outer diameter for a particular patient can make it difficult to navigate through the larynx and trachea, it may be more prone to irritation of the tracheal walls of the patient, and such endotracheal tube may increase the time it takes to intubate the patient. On the other hand, an endotracheal tube having too small a tube diameter may be associated with an increased work required for the patient to breathe properly. A standard feature in these endotracheal intubation devices is that the outer diameter of the tube remains constant throughout its' entire length.

Ever since the endotracheal tubes were introduced, anesthesiologists and other physicians have experienced difficulties with the intubation of endotracheal tubes into the trachea. Of all procedures done in the intensive care unit related to maintaining ventilation of critically ill patients, intubation of the trachea has been a procedure associated with the greatest number of complications. For example, the patient is typically temporarily paralyzed to facilitate visualization of the vocal cords by relaxing the jaw muscles and preventing the patient from retching or otherwise interfering with the placement of a laryngoscope down the patients' throat. This process eliminates the contribution that the patient might take towards their own breathing. Failure to place the endotracheal tube rapidly can result in death if the patient cannot be properly ventilated.

What has not been appreciated to date is an intubation device with a variable outer diameter at certain portions along its length, and that by providing and using such a novel device the intubation procedure can be made much more simple and fail safe from operational errors. What is needed is an endotracheal tube intubation device that when used reduces complications during esophageal intubation or prevents unwanted endobranchial intubation. A common problem that occurs with prior art intubation devices, in particular whenever a patient is oriented in anything other than a supine position while undergoing a procedure involving intubation, is kinking of the tube that interrupts the flow of air. Accordingly, there is also a need for a kink resistant intubation device.

What is needed is an intubation device having a variable or differential diameter between the first and second tube portions. The invention satisfies this need, and it has utility in a wide range of procedures and hospital settings including but not limited to ICU, CCU, and operating rooms. Our novel intubation tube and device, for the first time ever, incorporates unique design elements that prevents or drastically reduces the occurrence of any of the above mentioned complications during an intubation. By reducing the incidence of improper placement and over extension of the tube into a patient's trachea in a manner prone to irritate surrounding tissues, the invention reduces the incidence of complications and patient discomfort resulting from operational errors that existing intubation devices are susceptible resulting from their design limitations. And it does so while requiring a much lower standard of expertise required of practitioners for prior art intubation devices.

### BRIEF SUMMARY

The inventions described and claimed herein have many attributes and embodiments including, but not limited to, those set forth or described or referenced in this Brief Summary. The inventions described and claimed herein are not limited to, or by, the features or embodiments identified in this Summary, which is included for purposes of illustration only and not restriction.

The inventions described and claimed herein relate to a kink resistant endotracheal tube intubation device and a method for using the device. The kink resistant endotracheal tube intubation device has a first tube portion in operable connection with a second tube portion. The first and second tube portions of the endotracheal tube intubation may be integrated into one piece or formed of two or more portions that are in operable attachment. The first tube portion is preferably substantially rigid and it has a lumen for dispensing gas or fluids, for example to anesthetize a patient.

The second tube portion of the kink resistant endotracheal tube intubation device is substantially flexible and kink resistant, and, like the first tube portion, the second tube portion of the endotracheal tube intubation device also has a lumen for dispensing gas or fluids. The second tube portion typically has an internal guide for assisting an operator to achieve a desired positioning of the second tube portion within a patients' mouth.

Another feature of the second tube portion is that in typical embodiments the second tube portion of the endotracheal tube intubation device has a diameter that is larger than the first tube portion. The variance in diameter functions to stop the tube from further insertion once it reaches the vocal cords of a patient. The variance in diameter also acts to ensure an endobranchial intubation does not occur where an endotracheal intubation is desired. The anesthesiologist, or other practitioner even if inexperienced, will stop the further insertion of the tube and will fix it at the point of entry (patient's mouth) thus preventing an esophageal intubation. Specifically, in some embodiments of the endotracheal tube intubation device, the second tube portion is between about 0 5 mm and about 10 mm larger than the diameter of the first tube portion, for example where the second tube portion is about 0.5 mm, 1 mm, 2 mm, 3 mm, 4 mm, 5 mm, 6 mm, 7 mm, 8 mm, 9 mm or 10mm larger than the diameter of the first tube portion.

It is desirable for the second tube portion of the endotracheal tube intubation device to be flexible. In preferred embodiments of the endotracheal tube intubation device, the second tube portion has a spiral, corrugated, ribbed or similar such design that provides flexibility for the second tube portion. The second tube portion may be a flexible extension of the first tube portion. The second tube portion of the endotracheal tube intubation device is not only flexible, but also has rigidity and the ability to change the conformation of the second tube portion by the action of the internal guide wire. The internal guide wire of the second tube portion is typically made from a metal or alloy, such as a non-toxic metal. The internal guide wire of the second tube portion can be disposed on the inner surface or the outer surface of the second tube portion, or alternatively embedded in the material comprising the second tube portion. More typically, the internal guide is disposed on an inner surface of the second tube portion or is integral with an inner surface of the second tube portion. However, the particular placement of the guide wire relative to the second tube portion is not critical so long as functionality is achieved.

It is often desirable to provide a seal between the outside of the tube or device and the interior of the passage in which the tube or device is inserted. With such a seal, substances can only flow through the passage via the tube or other medical device. This allows a physician, nurse, or other medical professional to have control over the type and amount of substances flowing into and out of the patient. To provide such a seal, inflatable cuffs are sometimes used in conjunction with such tubes to seal these types of tracheal tubes. These cuffs can generally be expanded into the surrounding trachea to seal the tracheal passage around the circumference of the tube.

Preferred embodiments of the kink resistant endotracheal tube intubation device are intended for use in an endotracheal intubation procedure and the second tube portion has a design that reduces the potential for kinking of the tube when in use. In use, the unique difference in diameter between the first tube portion and the second tube portion of the invention provides a signal and an indication of the position of the device within a patient. The difference in diameter between the first tube portion and the second tube portion allows the endotracheal tube intubation device to be repositioned easily by a physician performing the procedure. This difference in diameter between the first tube portion and the second tube portion additionally provides an alert for the physician that the tube has reached the vocal cord when an endotracheal intubation is desired. When used according to methods provided herein, the novel difference in diameter between the first tube portion and the second tube portion of the kink resistant endotracheal tube intubation device reduces the incidence of aspiration pneumonia during use.

Also provided is a method of endotracheal intubation. The method generally encompasses selecting a patient in need of endotracheal intubation and selecting a kink resistant endotracheal tube intubation device provided herein. A physician inserts the endotracheal tube intubation device through the mouth and pharynx of a patient to perform the procedure.

In use, the difference in diameter between the first tube portion and the second tube portion of said kink resistant endotracheal tube intubation device provides an indication of the position of the device within a patient. The difference in diameter between the first tube portion and the second tube portion of the kink resistant endotracheal tube intubation device also allows the endotracheal tube intubation device to be repositioned easily during use. The difference in diameter between the first tube portion and the second tube portion of the kink resistant endotracheal tube intubation device additionally provides an alert that the tube has reached the vocal cord when an endotracheal intubation is desired. The difference in diameter between the first tube portion and the second tube portion of said kink resistant endotracheal tube intubation device reduces the incidence of aspiration pneumonia during use.

### BRIEF DESCRIPTION OF THE DRAWINGS

FIG. 1 shows a front view of an embodiment of a kink resistant endotracheal tube intubation device.

FIG. 2 is a cross-sectional view of the embodiment of a kink resistant endotracheal tube intubation device shown in FIG. 1 taken along axis 2 shown in FIG. 1.

FIG. 3 is a cross-sectional view of the embodiment of a kink resistant endotracheal tube intubation device shown in FIG. 2 taken along axis 3 shown in FIG. 2.

FIG. 4 is a cross-sectional view of the embodiment of a kink resistant endotracheal tube intubation device shown in FIG. 2 taken along axis 4 shown in FIG. 2.

FIG. 5 shows a perspective view of an embodiment of a kink resistant endotracheal tube intubation device.

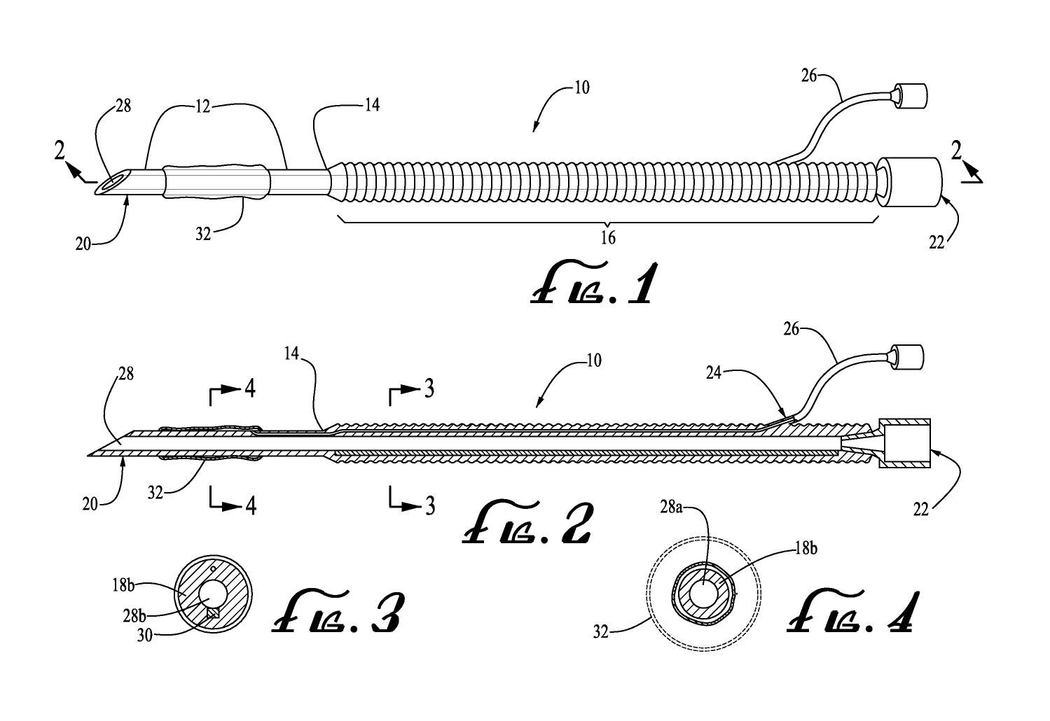

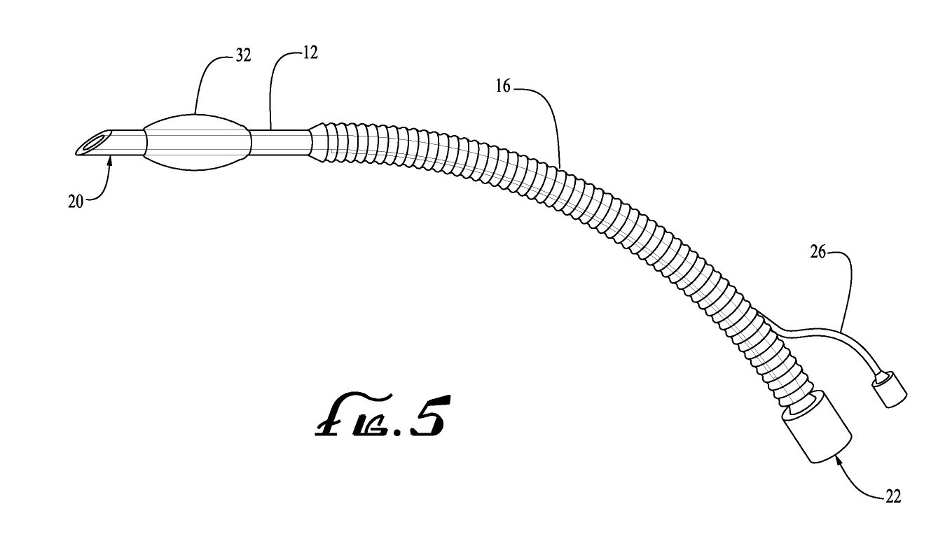

### DETAILED DESCRIPTION

Various aspects of the invention will now be described with reference to the following experimental section which will be understood to be provided by way of illustration only and not to constitute a limitation on the scope of the invention.

The kink resistant endotracheal tube intubation device 10 has a first tube portion 12 in operable connection with a second tube portion 16. The first tube portion 12 is proximal to a patients' mouth or nose (proximal end 20) upon insertion and intubation of the patient, while the second tube portion 16 is distal to the place insertion into the patient (distal end 22). The kink resistant endotracheal tube intubation device 10 further includes an intermediate portion 14 that connects the first and second tube portions (12, 16 respectively). The kink resistant endotracheal tube intubation device 10 has a length measured along its longitudinal axis that may vary in different embodiments. The kink resistant endotracheal tube intubation device 10 may be adapted to connect to a connector, tube, another device, or the like through a port 24 or opening in the outer wall 18 along the longitudinal axis at various portions of the first, second, or intermediate tube portions. A flexible attachment 26 connected to the port 24 can be used, for example, to connect to a reservoir (not shown) containing selected fluids. The distal end 22 of the second tube portion 16 may also be configured to be suitable for connection to other medical devices or systems, such as a ventilator (not shown).

The first and second tube portions (12, 16) of the endotracheal tube intubation device 10 may be integrated into one piece or formed of two or more portions that are in operable attachment at the intermediate portion 14. The first tube portion 12 is preferably substantially rigid and is more rigid than the second tube portion 16, and both portions have a lumen 28 for dispensing gas or fluids, for example to anesthetize a patient. The relative rigidity of the first tube portion 12 facilitates guiding the kink resistant endotracheal tube intubation device 10 through the patient's larynx and trachea. Suitable materials for the second tube portion 16 be polyvinyl chloride (PVC) or related materials, and may also include for example polyethylene, silicone, rubber, polyurethane, and similar materials. It may also be desirable to include a radio opaque material that makes the endotracheal tube intubation device 10 more visible on a chest X Ray. Generally, the first tube portion 12, intermediate tube portion 14, and second tube portion 16 may be attached by any suitable means whereby the outer wall 28b of the second tube portion 14 is attached to the outer wall 28a of the first tube portion 12, such as by adhesives or heat sealing, solvent bonding, RF sealing, ultrasonic welding. Alternatively, the kink resistant endotracheal tube intubation device 10 may be extruded as a single tube and the first tube portion 12, intermediate tube portion 14, and second tube portion 16 may be one integral piece where the outer wall 18 is one contiguous piece.

In the art, the “size” of an endotracheal tube refers to its internal diameter (ID). Thus as an example a “size 6” endotracheal tube is one with an internal diameter of 6 mm Both the internal diameter and the external diameter of an endotracheal tube intubation device 10 are important measurements that may be varied according to the particular patients requirements. Generally, the internal diameter (ID) will have an affect on the resistance and fluid/air flow and the outer diameter is selected and chosen to accommodate a particular patients' size requirements (for example if the patient is an infant or child). An important aspect of the invention, described in greater detail below, is that while the internal diameter (ID) of endotracheal tube intubation device 10 may remain unchanged over the length of the device, the endotracheal tube intubation device 10 may have a outer diameter that varies at different portions along the length of the device. Accordingly, endotracheal tube intubation devices 10 are provided with different primary internal diameters (IDs) and, importantly, different primary outer diameters such that each individual embodiment has at least two different outer diameters along its longitudinal axis.

The first tube portion 12 of the kink resistant endotracheal tube intubation device 10 has a lumen 28a defined by the internal diameter (ID) that in operable flow-through connection with a corresponding second tube portion lumen 28b. Preferably there is a substantially seamless connection between the first portion lumen 28a and the second tube portion lumen 28b in order to reduce resistance of the flow of gas or liquids through the of the kink resistant endotracheal tube intubation device 10. An endotracheal tube intubation device 10 according to the invention may use any ID (internal diameter) size device found in the art. An inflatable cuff 32 is typically included as part of the first tube portion 12 and it is typically disposed near the proximal end 20 of the first tube portion 12 such that the inflatable cuff 32 is closer to the proximal end 20 and is typically disposed between that the transition between the first tube portion 12 and the second tube portion 16. As with other prior art devices, the inflatable cuff 32 can be used to facilitate a seal between the device and the patients' airways.

The second tube portion 16 of the kink resistant endotracheal tube intubation device 10 is substantially flexible and kink resistant, and, like the first tube portion 12, the second tube portion 16 of the endotracheal tube intubation device 10 has a second tube portion lumen 28b for dispensing gas or other fluids. The second tube portion 16 typically may include an internal guide wire 30, passing through second tube portion lumen 28b or integrated with the outer wall 18b of the second tube portion 16. The internal guide wire 30 of second tube portion 16 functions to provide rigidity and operational control to the second tube portion 16 useful for assisting an operator to achieve a desired positioning of the second tube portion 16 within a patient. The internal guide wire 30 may also be integrated with or pass through the first tube portion 12.

A suitable outer diameter for the kink resistant endotracheal tube intubation device 10 may be for example between about 2.5 mm and about 20 mm, and more typically between about 2.5 mm and about 13 mm. As discussed below, the outer diameter of the first tube portion 12 of the kink resistant endotracheal tube intubation device 10 is preferably designed to be slightly less than the outer diameter of the second tube portion 16 of the kink resistant endotracheal tube intubation device 10. In some embodiments of the endotracheal tube intubation device 10, the diameter of the second tube portion 16 is between about 0.5 mm and about 10 mm larger than the diameter of the first tube portion 12, and more typically the diameter of the second tube portion 16 is between about 0.5 mm and about 3 mm larger than the diameter of the first tube portion 12. In other embodiments of the endotracheal tube intubation device 10, the second tube portion 16 is about 0.5 mm, 1 mm, 2 mm, 3 mm, 4 mm, 5 mm, 6 mm, 7 mm, 8 mm, 9 mm or 10 mm larger than the diameter of the first tube portion 12. In one preferred embodiment, the second tube portion 16 is about 1 mm larger than the diameter of the first tube portion. Embodiments where the second tube portion 16 is 0.6 mm, 0.7 mm, 0.8 mm, 0.9 mm, 1.1 mm, 1.2 mm, 1.3 mm, 1.4 mm, 1.5 mm larger, or between 1.5 mm and 2.0 mm larger than the diameter of the first tube portion 12 are contemplated.

The intermediate tube portion 14 of the endotracheal tube intubation device 10, where the transition between the first tube portion 12 and second tube portion 16 occurs, is where this diameter change takes place, and it is typically between about 5 cm and about 15 cm from the tip of the proximal end 20 of the first tube portion 12. In preferred embodiments, the intermediate tube portion 14 of the endotracheal tube intubation device 10, where the transition between the first tube portion 12 and second tube portion 16 diameter change takes place, is between about 7 cm and about 10 cm from the tip of the proximal end 20 of the first tube portion 12. In certain embodiments, this change in diameter is rather abrupt and appears as a step up in diameter in cross-section. In other embodiments, this change in diameter may be more gradual along a length, it is generally desirable to have the change in desire occur over a short span in length about the longitudinal axis. Even as such, there may be a slight taper or bevel in the outer diameter transition in certain embodiments. This unique difference in diameter between the first and second tube portions (12, 16) provides the operator an indication alert during an intubation. The variance in diameter functions to stop the tube from further insertion once it reaches the vocal cords of a patient thus ensuring an endotracheal intubation where desired. While performing an intubation procedure using a device according to the invention, the operator, even if inexperienced, will stop the further insertion of the tube and will fix it at the point of entry (patient's mouth) thus preventing an esophageal intubation.

It is desirable for the second tube portion 16 of the endotracheal tube intubation device 10 to be flexible. In certain embodiments of the endotracheal tube intubation device 10, the second tube portion 16 has a spiral or corrugated design that provides both strength and flexibility for the second tube portion 16. In other embodiments of the endotracheal tube intubation device 10, both the first tube portion 12 and the second tube portion 16 have a spiral or corrugated design feature. In still other embodiments of the endotracheal tube intubation device 10, neither or the first tube portion 16 or the second tube portion 16 have a spiral or corrugated design feature. In a preferred embodiment, the second tube portion 16 is a flexible extension of the first tube portion 12.

The second tube portion 16 of the endotracheal tube intubation device 10 is not only flexible, but also has rigidity and the ability to change the conformation of the second tube portion 16 by the action of an internal guide wire 30. The internal guide wire 30 is typically made from a metal or alloy, such as a non-toxic metal and it may further comprise a coating material. The internal guide wire 30 of the second tube portion 16 can be disposed on the inner surface or the outer surface of the second tube portion 16, or alternatively embedded in the material comprising the second tube portion or the outer wall 18b. Alternatively, the internal guide wire 30 is disposed on an inner surface of the second tube portion 16 or is integral with an inner surface of the second tube portion 16. In other embodiments, both the first tube portion 12 and the second tube portion 16 have the internal guide wire 30. In other embodiments, the internal guide wire 30 is an independent element capable of inserting through the lumen 28 of one or more of the first tube portion 12 and the second tube portion 16 have the internal guide wire 30 of the endotracheal tube intubation device 10.

Preferred embodiments of the kink resistant endotracheal tube intubation device 10 are intended for use in an endotracheal intubation procedure, and accordingly the second tube portion 16 has a design that reduces the potential for kinking of the tube when in use. In use, the unique difference in diameter between the first tube portion 12 and the second tube portion 16 of the invention provides a signal and an indication of the position of the device 10 within a patient. The difference in diameter between the first tube portion 12 and the second tube portion 16 allows the endotracheal tube intubation device 10 to be repositioned easily by a physician performing the procedure. This difference in diameter between the first tube portion 12 and the second tube portion 16 additionally provides an alert for the physician that the tube has reached the vocal cord when an endotracheal intubation is desired. When used according to methods provided herein, the novel difference in diameter between the first tube portion 12 and the second tube portion 16 of the kink resistant endotracheal tube intubation device 10 reduces the incidence of aspiration pneumonia during use.

Also provided is a method of endotracheal intubation. The method generally encompasses selecting a patient in need of endotracheal intubation and selecting a kink resistant endotracheal tube intubation device 10 provided herein. A physician inserts the endotracheal tube intubation device 10 through the mouth and pharynx of a patient to perform the procedure. In use, the difference in diameter between the first tube portion 12 and the second tube portion 16 of the kink resistant endotracheal tube intubation device 10 provides an indication of the position of the device within a patient. The difference in diameter between the first tube portion 12 and the second tube portion 16 of the kink resistant endotracheal tube intubation device also allows the endotracheal tube intubation device to be repositioned easily during use. The difference in diameter between the first tube portion 12 and the second tube portion 16 of the kink resistant endotracheal tube intubation device additionally provides an alert that the tube has reached the vocal cords when an endotracheal intubation is desired. The difference in diameter between the first tube portion 12 and the second tube portion 16 of the kink resistant endotracheal tube intubation device reduces the incidence of aspiration pneumonia during use.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

What is claimed is:

1. An kink resistant endotracheal tube intubation device comprising:
a first tube portion for dispensing a gas to anesthetize a patient, wherein said first tube portion is substantially rigid and has a lumen for dispensing gas, and
a second tube portion in operable connection with the first tube portion, wherein the second tube portion is substantially flexible and kink resistant, wherein the second tube portion further comprises a lumen for dispensing gas and an internal guide for assisting an operator to achieve a desired positioning of the second tube portion within a patients' mouth,
wherein the second tube portion has an outer diameter that is larger than the outer diameter of said first tube portion.

2. An endotracheal tube intubation device of claim 1, wherein the outer diameter of the second tube portion is between about 0.5 mm and about 10 mm larger than the outer diameter of the first tube portion.

3. An endotracheal tube intubation device of claim 1, wherein the outer diameter of the second tube portion is between about 0.5 mm and about 3 mm larger than the outer diameter of the first tube portion.

4. An endotracheal tube intubation device of claim 1, wherein the outer diameter of the second tube portion is about 1 mm larger than the outer diameter of the first tube portion.

5. An endotracheal tube intubation device of claim 1, wherein the second tube portion has a spiral design that provides flexibility for the second tube portion.

6. An endotracheal tube intubation device of claim 6, wherein the second tube portion is a flexible extension of the first tube portion.

7. An endotracheal tube intubation device of claim 1, wherein the internal guide comprises a non-toxic metal.

8. An endotracheal tube intubation device of claim 1, wherein the internal guide is disposed on an inner surface of the second tube portion.

9. An endotracheal tube intubation device of claim 1, wherein the internal guide is integral with an inner surface of the second tube portion.

10. An endotracheal tube intubation device of claim 1 for use in an endotracheal intubation procedure.

11. An endotracheal tube intubation device of claim 1, wherein the second tube portion has a design that reduces the potential for kinking of the tube when in use.

12. An endotracheal tube intubation device of claim 1, wherein the difference in outer diameter between the first tube portion and the second tube portion provides an indication of the position of the device within a patient.

13. An endotracheal tube intubation device of claim 2, wherein the difference in outer diameter between the first tube portion and the outer diameter of the second tube portion allows the endotracheal tube intubation device to be repositioned easily during use.

14. An endotracheal tube intubation device of claim 2, wherein the difference in outer diameter between the first tube portion and the second tube portion provides an alert that the tube has reached the vocal cord when an endotracheal intubation is desired.

15. An endotracheal tube intubation device of claim 2, wherein the difference in outer diameter between the first tube portion and the second tube portion reduces the incidence of aspiration pneumonia during use.

16. A method of endotracheal intubation, the method comprising the following steps:
a) selecting a patient in need of endotracheal intubation;
b) selecting a kink resistant endotracheal tube intubation device, the device comprising:
a first tube portion for dispensing a gas to anesthetize a patient, wherein the first tube portion is substantially rigid and has a lumen for dispensing gas, and
c) a second tube portion in operable connection with the first tube portion and having a outer diameter that is larger than the outer diameter of the first tube portion, wherein the second tube portion is substantially flexible and kink resistant and the second tube portion further comprises a lumen for dispensing gas and an internal guide for assisting an operator to achieve a desired positioning of the second tube portion within a patients' mouth, and wherein the difference in outer diameter between the first tube portion and the second tube portion provides an indication and warning that prevents unwanted intubation, and
d) inserting the endotracheal tube intubation device through the mouth and pharynx of a patient and intubating said patient.

17. A method of endotracheal intubation of claim 16, wherein the difference in outer diameter between the first tube portion and the outer diameter of the second tube portion of said kink resistant endotracheal tube intubation device provides an indication of the position of the device within a patient.

18. A method of endotracheal intubation of claim 16, wherein the difference in outer diameter between the first tube portion and the second tube portion of said kink resistant endotracheal tube intubation device allows the endotracheal tube intubation device to be repositioned easily during use.

19. A method of endotracheal intubation of claim 16, wherein the difference in outer diameter between the first tube portion and the second tube portion of said kink resistant endotracheal tube intubation device provides an alert that the tube has reached the vocal cord when an endotracheal intubation is desired.

20. A method of endotracheal intubation of claim 16, wherein the difference in outer diameter between the first tube portion and the second tube portion of said kink resistant endotracheal tube intubation device reduces the incidence of aspiration pneumonia during use.
